(19) United States
(12) Patent Application Publication
(10) Pub. No.: US 2021/0330192 A1
(43) Pub. Date: Oct. 28, 2021

(54) HOLDER AND EAR THERMOMETER USING THE HOLDER

(71) Applicant: BIO ECHO NET inc., Hokkaido (JP)

(72) Inventor: Hideki TANAKA, Hokkaido (JP)

(73) Assignee: BIO ECHO NET inc

(21) Appl. No.: 17/372,326

(22) Filed: Jul. 9, 2021

Related U.S. Application Data
(63) Continuation of application No. PCT/JP2019/051144, filed on Dec. 26, 2019.

(30) Foreign Application Priority Data
Jan. 10, 2019 (JP) 2019-002521

(51) Int. Cl. A61B 5/01; A61B 5/00; A61B 46/17

(52) U.S. Cl. CPC A61B 5/01; A61B 2560/0406; A61B 46/17; A61B 5/6817

(57) ABSTRACT

A holder, for an ear thermometer including an infrared sensor for measuring a temperature of an eardrum of an ear of a temperature measurement target person in a non-contact manner and configured to be worn to an ear hole of the temperature measurement target person, includes a holder body configured to be attached so as to cover at least a part of the ear thermometer, a flexible engaging arm curved outward and extended from the holder body and configured to be engageable with an ear concha of the temperature measurement target person, and a flexible support portion extending from the holder body to join and support the engaging arm.

### CROSS REFERENCE TO RELATED APPLICATIONS

This application is a continuation of International Application No. PCT/JP2019/051144, filed on Dec. 26, 2019, and based upon and claims the benefit of priority from Japanese Patent Application No. 2019-002521, filed on Jan. 10, 2019, the entire contents of all of which are incorporated herein by reference.

### TECHNICAL FIELD

The disclosure relates to a holder for holding a thermometer in an ear for measuring a body temperature of a temperature measurement target person, and an ear thermometer using the holder.

### BACKGROUND

For example, in an operating room, an intensive care unit or the like, it is essential to measure a body temperature of a temperature measurement target person during the operation.

Further, for example, it may be necessary to measure a body temperature as part of physical condition management for workers who work for a long period of time with a large physical burden and athletes who play various sports.

Since it is necessary to continuously measure a body temperature of a temperature measurement target person such as a patient, a worker, or an athlete over a long period of time, it is important to reduce the burden on the body.

As a thermometer that meets such demands, an ear thermometer for measuring a temperature of an eardrum by inserting a probe into an ear hole of a temperature measurement target person has been proposed (see JP 503618 B2).

### SUMMARY

Here, an ear thermometer measures a body temperature by detecting infrared rays emitted from an eardrum with an infrared sensor.

Therefore, in order to accurately measure a body temperature, it is necessary to hold an ear thermometer in an ear canal so that a measurement portion of the infrared sensor faces the eardrum side.

However, depending on the movement of a head of a temperature measurement target person, the position of an ear thermometer may shift in an ear canal, and accordingly the measurement portion of the infrared sensor may not accurately point to the eardrum.

In particular, when a worker who performs various tasks or an athlete who engages in various sports wears an ear thermometer, there is a risk that the position of the ear thermometer will be greatly displaced in an ear canal due to vigorous movement, and accordingly there is an inconvenience that an accurate body temperature measurement cannot be performed.

The present application has been made in view of the above problems, and an object of the present application is to provide a holder capable of stably holding an infrared sensor in a state where the infrared sensor can efficiently receive infrared rays emitted from an eardrum, and an ear thermometer using the holder.

In order to achieve the above object, a holder according to an embodiment is a holder for an ear thermometer including an infrared sensor for measuring a temperature of an eardrum of an ear of a temperature measurement target person in a non-contact manner, and configured to be worn to an ear hole of the temperature measurement target person. The holder includes a holder body configured to attached so as to cover at least apart of the ear thermometer, a flexible engaging arm curved outward and extended from the holder body and configured to be engageable with an ear concha of the temperature measurement target person, and a flexible support portion extending from the holder body to join and support the engaging arm.

As a result, the holder of the ear thermometer can stably hold the ear thermometer in a state where infrared rays emitted from an eardrum can be efficiently received by the infrared sensor.

A flexible joint portion may be formed inside an intersection of the engaging arm and the support portion.

This makes it possible to improve the retentiveness of the ear thermometer in an ear by the holder of the ear thermometer.

A part of the engaging arm may be formed so as to be engageable with an ear cavum concha of the temperature measurement target person.

This makes it possible to further improve the retentiveness of the ear thermometer in an ear by the holder of the ear thermometer.

A tip portion that is cuttable from the engaging arm may be provided at a tip of the engaging arm.

As a result, since the holder of the ear thermometer accommodates ears of various sizes, this makes it possible to further improve the retentiveness of the ear thermometer.

A holder according to other embodiment is a holder for an ear thermometer including an infrared sensor for measuring a temperature of an eardrum of an ear of a temperature measurement target person in a non-contact manner and configured to be worn to an ear hole of the temperature measurement target person. The holder includes a holder body configured to be attached so as to cover at least a part of the ear thermometer, and a flexible engaging arm projecting outward from the holder body in an annular manner and configured to be engageable with an ear cavum concha of the temperature measurement target person.

As a result, since the holder of the ear thermometer can be shared by the left and right ears, this makes it possible to improve the convenience of the holder as well as the retentiveness of the ear thermometer.

An ear thermometer according to other embodiment is an ear thermometer using the holder according to the first or second aspect of the present invention.

As a result, by the function of the holder, the ear thermometer can be stably held in a state in which an infrared sensor can efficiently receive infrared rays emitted from an eardrum.

With each embodiment, it is possible to provide a holder capable of stably holding an ear thermometer in a state where infrared rays emitted from an eardrum can be efficiently received by an infrared sensor, and an ear thermometer using the holder.

### BRIEF DESCRIPTION OF THE DRAWINGS

FIG. 1 is a perspective view illustrating an ear thermometer to which a holder according to a first embodiment is attached.

FIG. 2 is a perspective view illustrating the ear thermometer from which the holder according to the first embodiment is removed.

FIG. 3 is a top view of the holder according to the first embodiment.

FIG. 4 is a side view of the holder according to the first embodiment.

FIG. 5 is a front view of the holder according to the first embodiment.

FIG. 6 is a bottom view of the holder according to the first embodiment.

FIG. 7A is a right side view illustrating a configuration example of the ear thermometer from which the holder according to the first embodiment is removed.

FIG. 7B is a bottom view illustrating a configuration example of the ear thermometer from which the holder according to the first embodiment is removed.

FIG. 7C is a front view illustrating a configuration example of the ear thermometer from which the holder according to a first embodiment is removed.

FIG. 8 is an explanatory view illustrating a state in which the ear thermometer to which the holder according to the first embodiment is attached is worn to an ear.

FIG. 9A is a side view of a holder according to a second embodiment.

FIG. 9B is a top view of the holder according to the second embodiment.

FIG. 9C is a front view of the holder according to the second embodiment.

FIG. 9D is a bottom view of the holder according to the second embodiment.

FIG. 10 is an explanatory view illustrating a state in which an ear thermometer to which the holder according to the second embodiment is attached is worn to an ear.

### DETAILED DESCRIPTION

### First Embodiment

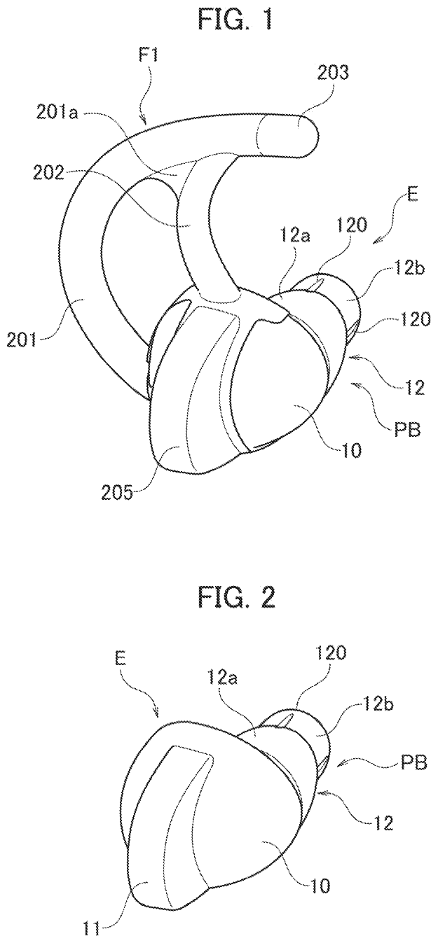

With reference to FIGS. 1 to 8, a holder F1 according to a first embodiment and an ear thermometer E using the holder F1 will be described.

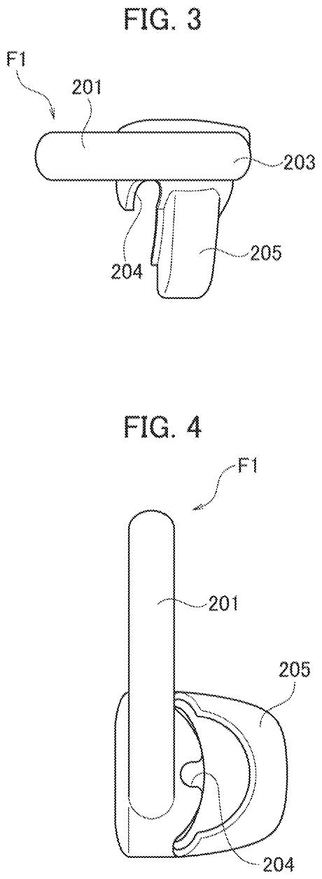

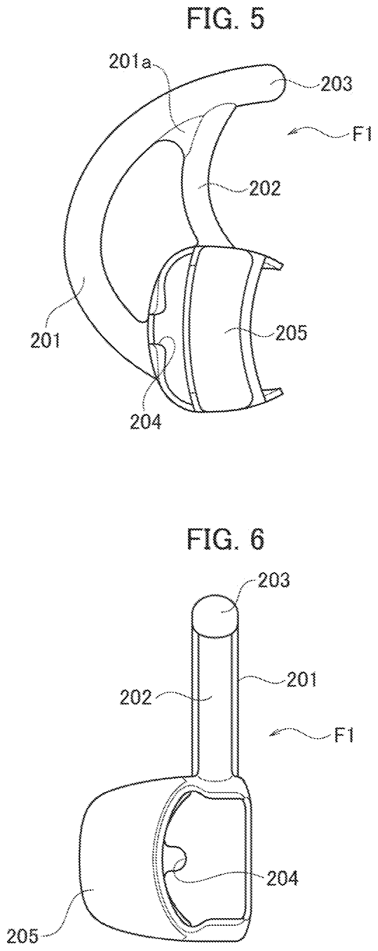

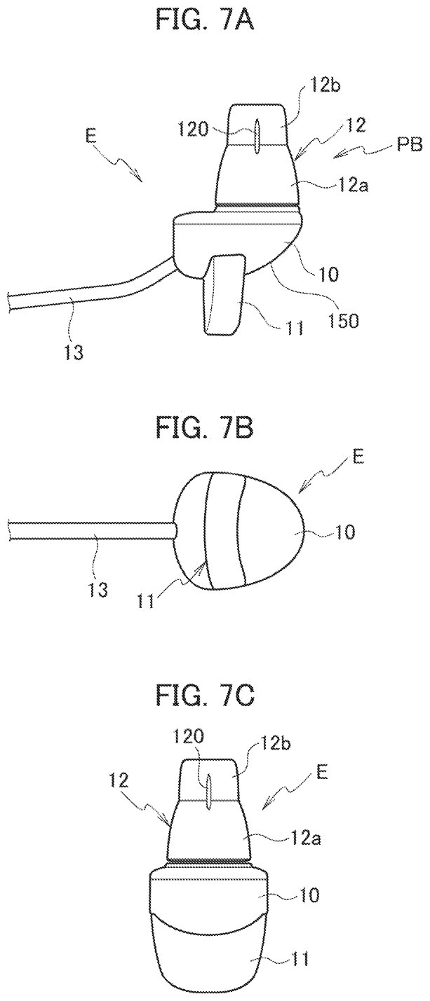

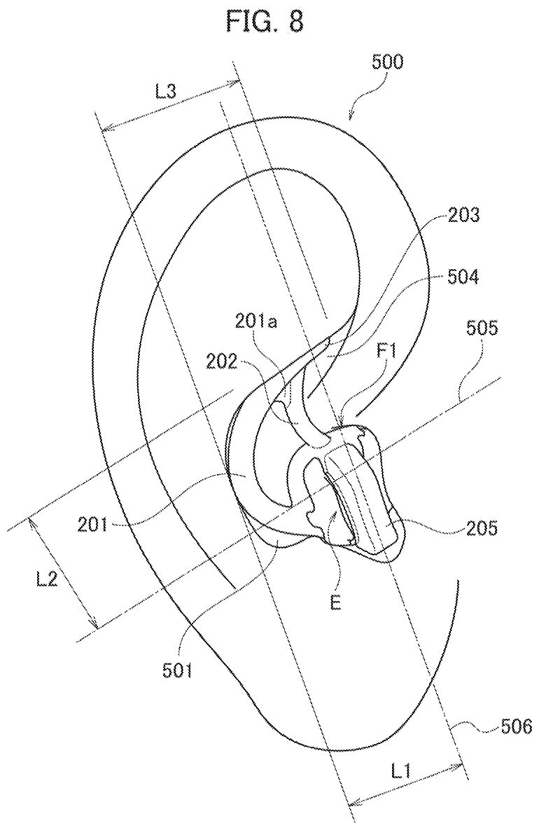

FIG. 1 is a perspective view illustrating the ear thermometer E to which the holder F1 according to the first embodiment is attached. FIG. 2 is a perspective view illustrating the ear thermometer E from which the holder F1 according to the first embodiment is removed. FIG. 3 is a top view of the holder F1. FIG. 4 is a side view of the holder F1. FIG. 5 is a front view of the holder F1. FIG. 6 is a bottom view of the holder F1. FIG. 7A is a right side view illustrating a configuration example of the ear thermometer E from which the holder F1 is removed, FIG. 7B is a bottom view of the same, and FIG. 7C is a front view of the same. FIG. 8 is an explanatory view illustrating a state in which the ear thermometer to which the holder is attached is worn to an ear.

(Configuration Example of Holder F1)

The holder F1 according to the first embodiment is a holder for the ear thermometer E including an infrared sensor (not illustrated) for measuring a temperature of an eardrum of an ear 500 of a temperature measurement target person such as a patient, a worker, and an athlete in a non-contact manner, and configured to be worn to an ear hole of the temperature measurement target person.

As illustrated in FIGS. 1 to 8, the holder F1 includes a holder body 205 configured to be attached so as to cover a part of a housing 10 of the ear thermometer E, a flexible engaging arm 201 extended from and curved outward the holder body 205 and engageable with an ear concha 504 (see FIG. 8) of a temperature measurement target person, and a flexible support portion 202 extending from the holder body 205 to join and support the engaging arm 201.

The holder F1 is integrally formed of a soft and flexible material such as silicone rubber.

Further, a flexible joint portion 201a is integrally formed inside an intersection of the engaging arm 201 and the support portion 202.

Further, a part of the engaging arm 201 is formed so as to be engageable with an ear cavum concha 501 of a temperature measurement target person.

Further, as illustrated in FIG. 1 and the like, a cuttable tip portion 203 may be provided at a tip of the engaging arm 201. As a result, it is possible to widely accommodate an ear of various size, which differs from person to person, and to further improve the retentiveness.

In FIG. 3 and the like, a reference numeral 204 is an insertion portion for a cable 13 of the ear thermometer E.

(Configuration Example of Ear Thermometer E)

As illustrated in FIGS. 7A to 7C, the ear thermometer E includes a probe PB including an infrared sensor (not illustrated in the drawings) for measuring a temperature of an eardrum of the ear 500 of a temperature measurement target person in a non-contact manner, and configured to be worn to an ear hole of the temperature measurement target person.

The probe PB mainly includes a probe body (not illustrated in the drawings) to be inserted into an ear hole of a temperature measurement target person, a housing 10 for supporting the probe body, and an in-ear type earpiece 12 configured to be attached to the probe body and come into contact inside the ear hole of the temperature measurement target person.

The housing 10 and the probe body are molded of ABS resin or the like. The housing 10 and the probe body may be integrally molded.

The infrared sensor is arranged in a recess formed on a tip side of the probe body.

The earpiece 12 includes a base portion 12a that partially exhibits a hollow conical shape and a substantially cylindrical tip portion 12b provided at one end of the base portion 12a and extending in a direction away from the housing 10.

The base portion 12a and the tip portion 12b are integrally formed of a soft and flexible material such as silicon rubber. A reference numeral 11 is a knob portion, and a reference numeral 120 is a hole through which an external sound or the like is passed.

The ear thermometer E having such a configuration can be stably held in an ear hole of a temperature measurement target person even by itself, but by attaching the holder F1 according to the first embodiment, the ear thermometer E can be more stably held in a state where the infrared emitted from an eardrum rays can be efficiently received by the infrared sensor.

(Example of Wearing)

With reference to FIG. 8, a state in which the ear thermometer E to which the holder F1 according to the first embodiment is attached is worn to an ear will be described.

As illustrated in FIG. 8, the engaging arm 201 of the holder F1 is engaged with the ear concha 504 of the right ear 500 of the temperature measurement target person. At this time, the engaging arm 201 is urged upward in the figure due to the elasticity of the support portion 202 and the joint portion 201a. As a result, the ear thermometer E is stably held in the right ear 500 of the temperature measurement target person.

Further, a part of the engaging arm 201 comes into contact with the ear cavum concha 501. As a result, the ear thermometer E is stably held by the right ear 500 of the temperature measurement target person.

Here, a size of the holder F1 will be described.

As illustrated in FIG. 8, when the distance from a vertical external ear hole center line 506 to the end of the ear cavum concha 501 is L1, when the distance from a lateral external ear hole center line 505 to the end of the ear concha 504 is L2, and when the distance from the end of the ear cavum concha 501 to the tip portion 203 of the engaging arm 201 is L3, by determining and combining the sizes of L1 to L3 according to the size of the ear, for example, three sizes of L, M, and S can be prepared.

The size L3 is designed to be slightly longer by providing the tip portion 203, and the tip portion 203 can be cut out according to the size of the ear, so that the size L3 can accommodate ears of various sizes without increasing the number of sizes.

Further, FIG. 8 illustrates a state in which the ear thermometer E to which the holder F1 is attached is worn to the right ear 500 of a temperature measurement target person, but it is also possible to prepare a left-ear wearing model in which an attachment state of the engaging arm 201 of the holder F1 is changed so that the model can be worn to a left ear.

### Second Embodiment

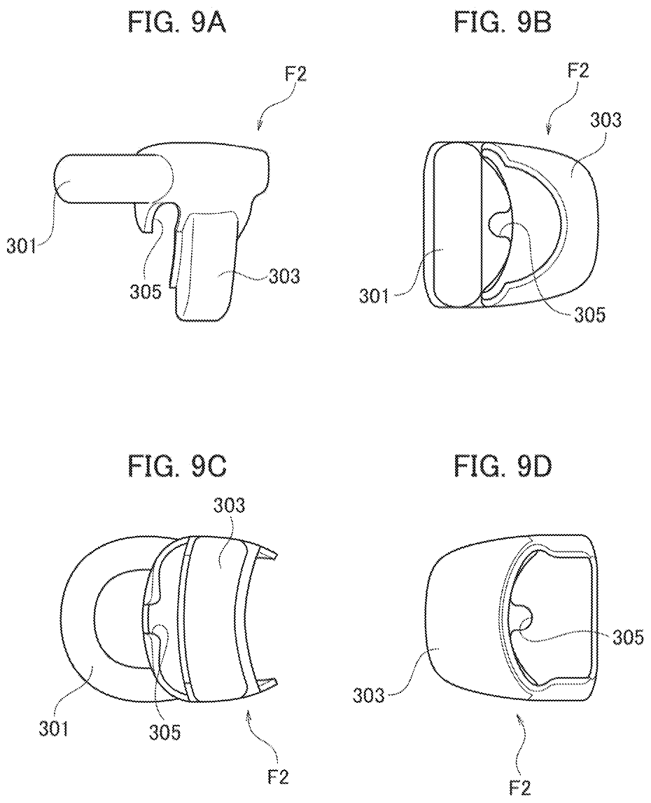

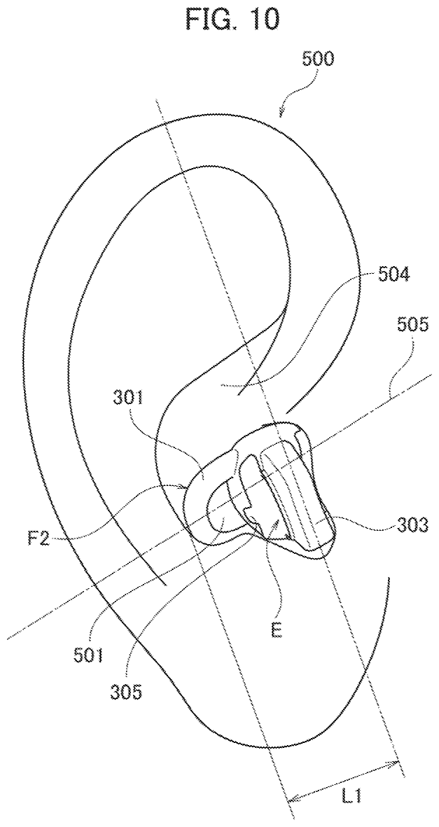

With reference to FIGS. 9 and 10, a holder F2 according to a second embodiment and the ear thermometer E using the holder F2 and will be described.

FIG. 9A is a side view illustrating the holder F2 according to the second embodiment, FIG. 9B is a top view of the same, FIG. 9C is a front view of the same, and FIG. 9D is a bottom view of the same. FIG. 10 is an explanatory view illustrating a state in which the ear thermometer E to which the holder F2 is attached is worn to an ear.

A holder according to the second embodiment is the holder F2 for an ear thermometer E including an infrared sensor (not illustrated in the drawings) for measuring a temperature of an eardrum of an ear 500 of a temperature measurement target person in a non-contact manner, and configured to be worn to an ear hole of the temperature measurement target person.

The holder F2 includes a holder body 303 configured to attached so as to cover at least a part of the housing of the ear thermometer, and a flexible engaging arm 301 projecting outward from the holder body 303 in an annular manner and configured to be engageable with the ear cavum concha 501 of the temperature measurement target person.

The holder F2 is integrally formed of a soft and flexible material such as silicone rubber.

In FIGS. 9A to 91, a reference numeral 305 is an insertion portion for the cable 13 of the ear thermometer E.

(Example of Wearing)

With reference to FIG. 10, a state in which the ear thermometer E to which the holder F2 according to the second embodiment is attached is worn to the ear will be described.

As illustrated in FIG. 10, a part of the engaging arm 301 of the holder F2 is engaged with the ear cavum concha 501. As a result, the ear thermometer E is stably held.

Since the holder F2 according to the second embodiment can be shared by the left and right ears, convenience can be further improved.

Further, by changing the distance L1 from the vertical external ear hole center line 506 to the end of the ear cavum concha 501, for example, three sizes L, M, and S can be prepared.

Although the holder of the ear thermometer according to the embodiments has been described based on the drawings, the present invention is not limited to these, and the configuration of each portion may be replaced with any configuration having the similar function.

What is claimed is:

1. A holder for an ear thermometer including an infrared sensor for measuring a temperature of an eardrum of an ear of a temperature measurement target person in a non-contact manner, and configured to be worn to an ear hole of the temperature measurement target person, the holder comprising:
a holder body configured to attached so as to cover at least a part of the ear thermometer,
a flexible engaging arm curved outward and extended from the holder body and configured to be engageable with an ear concha of the temperature measurement target person, and
a flexible support portion extending from the holder body to join and support the engaging arm.

2. The holder of claim 1, wherein a flexible joint portion is formed inside an intersection of the engaging arm and the support portion.

3. The holder of claim 1, wherein a part of the engaging arm is formed so as to be engageable with an ear cavum concha of the temperature measurement target person.

4. The holder of claim 1, wherein a tip portion that is cuttable from the engaging arm is provided at a tip of the engaging arm.

5. A holder for an ear thermometer including an infrared sensor for measuring a temperature of an eardrum of an ear of a temperature measurement target person in anon-contact manner, and configured to be worn to an ear hole of the temperature measurement target person, the holder comprising:
a holder body configured to be attached so as to cover at least a part of the ear thermometer,
a flexible engaging arm projecting outward from the holder body in an annular manner and is engageable with an ear cavum concha of the temperature measurement target person.

6. An ear thermometer configured to be worn to an ear hole of a temperature measurement target person, comprising:
an infrared sensor configured to measure a temperature of an eardrum of an ear of the temperature measurement target person in a non-contact manner; and
a holder comprising:
a holder body attached so as to cover at least a part of the ear thermometer,
a flexible engaging arm curved outward and extended from the holder body and configured to be engageable with an ear concha of the temperature measurement target person, and
a flexible support portion extending from the holder body to join and support the engaging arm.
